(19) United States
(12) Patent Application Publication
(10) Pub. No.: US 2026/0256863 A1
(43) Pub. Date: Sep. 3, 2026

(54) COMPOSITION, COMPRISING CINNAMOMUM CASSIA EXTRACT HAVING INNOVATIVE CKD EXTRACTION TECHNOLOGY (ICET) TECHNOLOGY APPLIED THERETO, FOR PREVENTING, ALLEVIATING, OR TREATING GASTRITIS OR PEPTIC ULCER

(71) Applicant: CHONG KUN DANG PHARMACEUTICAL CORP., Seoul (KR)

(72) Inventors: Junheon KIM, Yongin-si (KR); Bo Hee HONG, Yongin-si (KR); Min Kwan CHO, Yongin-si (KR); Min Soo KIM, Yongin-si (KR); Shin Jung PARK, Yongin-si (KR)

(21) Appl. No.: 18/846,760

(22) PCT Filed: Mar. 17, 2023
(86) PCT No.: PCT/KR2023/003614

§ 371 Date: Sep. 13, 2024

(30) Foreign Application Priority Data
Mar. 18, 2022 (KR) 10-2022-0034287
Sep. 5, 2022 (KR) 10-2022-0112210

(51) Int. Cl. A61K 36/54; A61P 1/04

(52) U.S. Cl. CPC A61K 36/54; A61P 1/04; A61K 2236/331; A61K 2236/35

(57) ABSTRACT

The present invention relates to a pharmaceutical composition comprising a Cinnamomum cassia extract as an active ingredient and is adapted to exhibit excellent prophylactic, palliative, or therapeutic effects on gastritis or peptic ulcers when administered at a predetermined dose.

### TECHNICAL FIELD

The present disclosure relates to a composition for preventing, improving, or treating gastritis or peptic ulcer, and more specifically to a composition for preventing, improving, or treating gastritis or peptic ulcer that includes Cinnamomum cassia extract to which innovative CKD extraction technology (iCet) is applied as an active ingredient and is administered in a specific dosage.

### BACKGROUND ART

A stomach is a part of digestive tract, a part inflated like a sack between esophagus and small intestine (duodenum), and an organ which stores food entered through the esophagus, breaks it down to facilitate digestion, and controls sending food to duodenum and harmonizes with secretion of digestive enzymes, thereby allowing efficient digestion and absorption. Factors that adversely affect a person's gastrointestinal function have extremely diverse properties, may occur in an upper gastrointestinal tract, a lower gastrointestinal tract or both, and include genetic, physiological, environmental, and mental factors, so that there is a wide range of gastrointestinal disorder factors. Representative diseases of the upper gastrointestinal tract are gastritis and peptic ulcer, which collectively indicates gastric ulcer and duodenal ulcer. The gastritis refers to damage and inflammation of gastric mucosa, and the gastric ulcer refers to the damage when such damage penetrates the mucosa and invades submucosa tissue and muscle layer. Further, the duodenal ulcer is an ulcer that occurs in the duodenum, and the gastric ulcer and the duodenal ulcer are collectively referred to as the peptic ulcer. Such gastritis and peptic ulcer are known to be caused by an imbalance between stomach acid, anti-inflammatory agents, and bacterial infections, which are called attack factors, and mucus, cell regeneration, alkaline secretion, etc., which are called defense factors.

As a treatment method for the gastritis and the peptic ulcer, antacids which neutralize excessively secreted gastric acid, histamine antagonists for suppressing acid secretion, proton pump inhibitors, anticholinergics, gastric mucosa protectants increasing resistance of stomach lining to digestive solution and helping recovery, etc. are the mainstream, and recently there are drug treatments that prescribe a combination of the above drugs and antibiotics to eliminate Helicobacter pylori. Characteristics of the antacids are fast-acting, and protection against damage of gastric mucosa from gastric acid through neutralizing gastric acid by raising pH in stomach. However, administration of inorganic substances may affect gastrointestinal tract smooth muscles, causing constipation, diarrhea, or allergic rejection.

In this regard, the present applicants have confirmed that the non-polar pretreatment of Cinnamomum cassia and the polar solvent extract have an outstanding prevention, improvement or treatment effect on the gastritis or the peptic ulcer, and these inventions were filed in Korean Patent Application Nos. 10-2017-0004418, 10-2020-0183904, and 10-2021-0147023.

### DISCLOSURE

### Technical Problem

Under the above background, the present disclosure is directed to providing a pharmaceutical composition with an outstanding inhibitory effect on gastritis or peptic ulcer by setting an appropriate dose of Cinnamomum cassia extract, and specifically, to providing a therapeutic composition for preventing, improving, or treating the gastritis or the peptic ulcer that includes the Cinnamomum cassia extract as an active ingredient and is administered in a specific daily dosage.

Further, the present disclosure is directed to providing a therapeutic composition for preventing, improving, or treating the gastritis or the peptic ulcer that includes the Cinnamomum cassia extract as an active ingredient and is administered in a specific single dosage.

### Technical Solution

In order to achieve the objectives, the present disclosure provides a pharmaceutical composition for preventing, improving, or treating gastritis or peptic ulcer that includes Cinnamomum cassia extract as an active ingredient, wherein a daily dosage is 210 to 360 mg based on the Cinnamomum cassia extract.

As an example, the pharmaceutical composition is provided in a single dosage of 70 to 120 mg based on the Cinnamomum cassia extract.

As an example, the pharmaceutical composition provides the Cinnamomum cassia extract including 5 mg/g or more of cinnamic acid.

As an example, the pharmaceutical composition is provided with an effective rate (FA set) on gastroscopy is 50% or more.

Further, the present disclosure provides a prevention, improvement, or treatment method of gastritis or peptic ulcer that administers Cinnamomum cassia extract to a subject in an amount of 210 to 360 mg per day.

As an example, the prevention, improvement, or treatment method of gastritis or peptic ulcer is provided in a single dosage of 70 to 120 mg based on the Cinnamomum cassia extract.

As an example, the prevention, improvement, or treatment method of gastritis or peptic ulcer is provided with the Cinnamomum cassia extract including 5 mg/g or more of cinnamic acid.

As an example, the prevention, improvement, or treatment method of gastritis or peptic ulcer is provided with an effective rate (FA set) on gastroscopy is 50% or more.

### Effect of the Invention

The present disclosure is configured so that Cinnamomum cassia extract as an active ingredient may be administered with a specific daily dosage and/or a single dosage and shows outstanding prevention, improvement, or treatment effect on gastritis or peptic ulcer, and therefore it is useful for development of pharmaceutical preparations for the above diseases.

### BRIEF DESCRIPTION OF THE DRAWINGS

FIG. 1 is a graph showing ulcer indices of an acute gastric damage animal model as a result of a test confirming an effect of suppressing gastric ulcer using the acute gastric damage animal model of Example 1.

FIG. 2 is a graph showing ulcer indices of an acute gastric damage animal model as a result of a test confirming an effect of suppressing gastric ulcer using the acute gastric damage animal model of Example 2.

FIG. 3 is a graph showing anti-inflammatory indices as a result of a test confirming an effect of suppressing gastric ulcer using the acute gastric damage animal model of Example 2.

### BEST MODE

Hereinafter, the present disclosure will be described in detail.

In an exemplary embodiment, the present disclosure relates to a pharmaceutical composition for preventing, improving, or treating gastritis or peptic ulcer that includes Cinnamomum cassia extract to which innovative CKD extraction technology (iCet) is applied as an active ingredient and having a daily dosage of 210 to 360 mg.

In another exemplary embodiment, the present disclosure provides prevention, improvement, or treatment method of gastritis or peptic ulcer that administers Cinnamomum cassia extract to which innovative CKD extraction technology (iCet) is applied to a subject in an amount of 210 to 360 mg per day.

In the present disclosure, ‘peptic ulcer’ collectively refers to duodenal ulcer and gastric ulcer. More specifically, it may be gastric ulcer.

In the present disclosure, Cinnamomum cassia is a Ranunculales Lauraceae evergreen broad-leaved tree belonging to a dicotyledon and is originated from China, is distributed in Sri Lanka, Indochina and Korea (Jeju), and refers to branches or bark of Cinnamomum cassia tree (Cinnamon tree) that grows up to about 8 m in height in mountain.

In the present disclosure, the term “Cinnamomum cassia extract” refers to an extract obtained by extracting from the Cinnamomum cassia. As a specific example, the Cinnamomum cassia extract may be prepared by drying the Cinnamomum cassia according to a conventional method, cutting or grinding the Cinnamomum cassia to an appropriate size for extraction, and extracting the same using an appropriate extraction solvent, and may be pretreated before extraction.

The extracting method may be performed using a known herbal extraction method such as hot water extraction, enfleurage extraction, reflux extraction or ultrasonic extraction, but is not limited thereto. Further, the extract may include not only a liquid extract itself, but also a diluted or concentrated solution of the extract, and a dried product obtained by drying the extract.

In an exemplary embodiment, the Cinnamomum cassia extract of the present disclosure may be an extract that is pretreated before extracting the Cinnamomum cassia with a polar solvent (i.e., a polar solvent extract of a pretreated Cinnamomum cassia).

In an exemplary embodiment, the pretreatment may be treatment of the Cinnamomum cassia with a non-polar solvent.

As a specific example, the non-polar solvent may be ethyl acetate.

The non-polar solvent may be used in a volume of 0.5 to 5 times, 0.7 to 4 times, or 1 to 3 times the weight of the Cinnamomum cassia (preferably dry weight), but is not limited thereto.

As a specific example, the pretreatment may be performed by immersing or flowing the cut or ground Cinnamomum cassia in the non-polar solvent such as ethyl acetate for 10 minutes to 5 hours, 20 minutes to 4 hours, or 30 minutes to 3 hours at a temperature of 20 to 35° C. or at room temperature.

In an exemplary embodiment, after the pretreatment is completed, the Cinnamomum cassia may be washed before extracting with the polar solvent.

Furthermore, the Cinnamomum cassia extract according to the present disclosure may be a polar solvent extract of the Cinnamomum cassia pretreated as described above and may be preferably a water extract.

The polar solvent used during extraction may be used in a volume of 5 to 12 times, 6 to 10 times, or 8 times the weight of the Cinnamomum cassia (preferably dry weight), but is not limited thereto.

As a specific example, the Cinnamomum cassia pretreated with the non-polar solvent may be extracted at a temperature of 70° C. or more, 70 to 100° C., or 80 to 100° C. Preferably, the Cinnamomum cassia pretreated with the non-polar solvent may be extracted with hot water at 70° C. or more. Further, with regards to extraction time, the extraction may be performed for 1 hour to 7 hours, 2 hours to 6 hours, or 5 hours. Such extraction may be performed once to several times, once to three times, or once to twice, and the extracted pretreated extract may be further filtered, concentrated, and/or dried, and as a method used at this time, filtration, concentration, and drying methods commonly used in the preparation of the extract may be used without limitation.

In the present disclosure, dry ext. (i.e., an extract in dry form) of the Cinnamomum cassia extract was used.

In an embodiment according to the present disclosure, the Cinnamomum cassia was dried and sliced to remove an ethyl acetate by adding 2 times a volume of ethyl acetate to the Cinnamomum cassia and soaking or rinsing for more than one hour at room temperature, and then a pretreated Cinnamomum cassia herb medicine was washed with water, and water corresponding to 8 times of the Cinnamomum cassia herb medicine was added to extract the Cinnamomum cassia for 5 hours at about 90° C., and this was repeated twice. The obtained extract was filtered, concentrated under reduced pressure, vacuum dried or spray dried to prepare a water extract pretreated with Cinnamomum cassia ethyl acetate.

The composition of the present disclosure shows an outstanding effect in preventing, improving, or treating the gastritis or the peptic ulcers such as gastric ulcer and duodenal ulcer by including the Cinnamomum cassia extract as described above.

In an example, in the pharmaceutical composition according to the present disclosure, a daily dosage is 210 to 360 mg. Preferably, the dosage may be 210 to 320 mg, 210 to 300 mg, 210 to 260 mg, 215 to 230 mg, or 225 mg.

When administering with such a daily dosage, it is preferable because it may show a remarkable effect of preventing, improving, or treating the gastritis or the peptic ulcer.

In an example, the daily dosage may be administered once or may be administered in a plurality of doses such as twice, three times, or four times.

In an example, in the pharmaceutical composition according to the present disclosure, a single dosage is 70 to 120 mg. Preferably, the dosage may be 70 to 100 mg or 70 to 80 mg. When administering with such a single dosage, it is preferable because it may show a remarkable effect of preventing, improving, or treating the gastritis or the peptic ulcer.

Meanwhile, the pharmaceutical composition provides the Cinnamomum cassia extract, which is an active ingredient in the pharmaceutical composition, including 5 mg/g or more of cinnamic acid.

In an example, when the pharmaceutical composition according to the present disclosure is administered, an effective rate (FA set) on gastroscopy is 50% or more.

The pharmaceutical composition of the present disclosure may be administered orally or parenterally and may be used in a form of general drug formulations. Preferable pharmaceutical formulations include oral administration formulations such as tablets, pills, powders, granules, hard or soft capsules, liquids, suspensions, etc., and these pharmaceutical formulations may be prepared using a conventional pharmaceutically acceptable carrier, for example, in case of oral administration formulations, excipients, binding agents, disintegrating agents, lubricants, solubilizers, suspending agents, preservatives or extenders, etc.

According to an example of the present disclosure, the Cinnamomum cassia extract according to the present disclosure reduces an area of gastric damage, reduces a gastric ulcer index, and inhibits inflammatory mediators (e.g., PGE2), and inhibits MPO when administered in an efficacy test for inducing acute gastric damage in rats, thereby showing an outstanding anti-inflammatory effect and also showing an outstanding effect of suppressing gastric ulcer. Further, when administered to acute and chronic gastritis patients, the complete cure rate was significantly higher than that of all control groups including a placebo group.

### DESCRIPTION OF EMBODIMENTS

### Examples

Hereinafter, the preset disclosure will be described in more detail through examples. The examples disclosed herein are merely for illustrating the present disclosure, and thus the scope of the present disclosure is not to be construed as limited by the examples.

### Preparation Example 1. Preparation of Cinnamomum cassia Extract According to the Present Disclosure

Specifically, a volume of 2 times of ethyl acetate based on the Cinnamomum cassia weight was added to the Cinnamomum cassia herbal medicine, and the Cinnamomum cassia herbal medicine was immersed at room temperature for more than 1 hour. After washing the Cinnamomum cassia herb medicine with water to remove ethyl acetate, a volume of 8 times of water based on the Cinnamomum cassia weight was added to the Cinnamomum cassia herbal medicine, and extract was extracted at around 90° C. for 5 hours (repeated twice). The extract was filtered, concentrated under reduced pressure, vacuum dried, or spray dried to prepare Cinnamomum cassia ethyl acetate pretreated water extract (extract obtained: 16 to 26->1; 16 to 26 kg of the Cinnamomum cassia herbal medicine was used to prepare 1 kg of final extract. That is, it means that the extract is concentrated to a weight of 1/16 to 1/26 of the weight of the Cinnamomum cassia herbal medicine).

### Example 1. Efficacy Test in an Acute Gastric Damage Animal Model of the Cinnamomum cassia Extract According to the Present Disclosure

To confirm the inhibition of gastric ulcer by the Cinnamomum cassia extract prepared in the Preparation Example 1, an efficacy test in an acute gastric damage animal model was conducted by the following method.

Specifically, the test was performed using 8-week-old SD rats. The test substance was administered as a single dose after fasting for 48 hours, and gastric ulcer was induced by administering Indomethacin 80 mg/kg (orally administered 30 minutes after administering the test substance). Each test group was divided into an induced group, a ranitidine (inhibition of histamine H2 receptor action) administration group, a rebamipide (gastric mucosa protectant) administration group, an artemisia extract (gastric mucosa protective agent) administration group, and a Cinnamomum cassia dry ext. (dry extract) administration group according to the present disclosure. Specifically, each group was divided as shown in Table 1 below.

TABLE 1

| Group | Dose(concentration) |
|---|---|
| G0 | Induced group (Vehicle control) |
| G1~G6 | Ranitidine 1, 3, 7, 15, 25, 50 mg/kg |
| G7~G13 | Rebamipide 1, 3, 7, 15, 25, 50, 100 mg/kg |
| G14~G19 | Artemisia extract 1, 3, 7, 15, 25, 50 mg/kg |
| G20~G26 | Cinnamomum cassia extract according to the |
| | present disclosure 1, 3, 7, 15, 25, 50, 100 mg/kg |

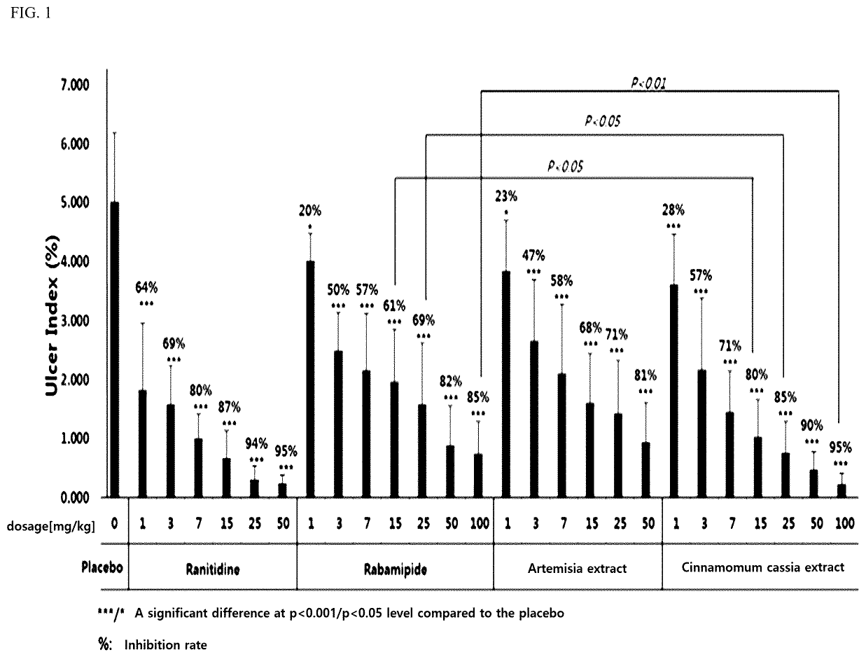

The results are shown in FIG. 1. As can be seen in FIG. 1, in the efficacy test for inducing acute gastric damage in rats, it was confirmed that an area of gastric damage was reduced in a dose-dependent manner when the dry extract of Cinnamomum cassia was administered at 1 to 100 mg/kg. Furthermore, in particular, when administering 100 mg/kg of the dry extract of Cinnamomum cassia, it showed the highest gastric damage inhibition rate, which was lowered to 95% compared to an ulcer index of placebo, and it was confirmed that this is a similar level to when administering 50 mg/kg of ranitidine.

### Example 2. Efficacy Test in an Acute Gastric Damage Animal Model According to a Dose of the Cinnamomum cassia Extract According to the Present Disclosure

To confirm the inhibition of gastric ulcer according to the dose of the Cinnamomum cassia extract prepared in the Preparation Example 1, an efficacy test in an acute gastric damage animal model was conducted by the following method.

Specifically, the test was performed using 8-week-old SD rats. The test substance was administered as a single dose after fasting for 48 hours, and gastric ulcer was induced by administering Indomethacin 80 mg/kg (orally administered 30 minutes after administering the test substance). Each test group was divided into a normal group, an induced group, an artemisia extract administration group, a rebamipide administration group, and a Cinnamomum cassia dry ext. (dry extract) administration group according to the present disclosure. Specifically, each group was divided as shown in Table 2 below.

TABLE 2

| Group | Dose(concentration) |
|---|---|
| G1 | Normal group (Vehicle control) |
| G2 | Induced group (Vehicle control) |
| G3 | Artemisia extract 50 mg/kg |
| G4 | Rebamipide 50 mg/kg |

| | | |
|---|---|---|
| G5 | Cinnamomum cassia extract | 100 mg/kg |
| G6 | according to the | 200 mg/kg |
| G7 | present disclosure | 400 mg/kg |

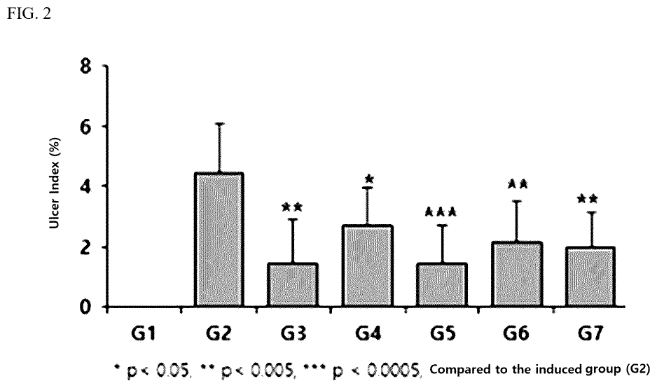

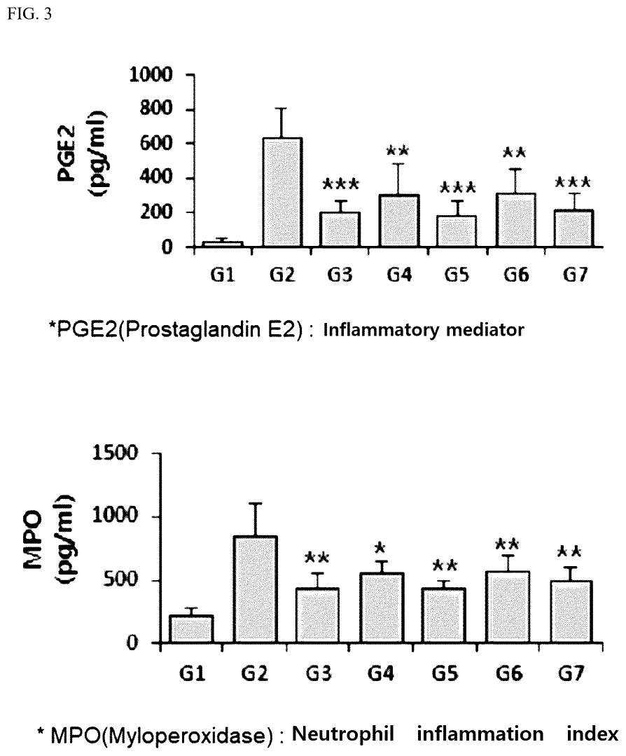

The results are shown in FIGS. 2 and 3.

As can be seen in FIGS. 2 and 3, in the efficacy test for inducing acute gastric damage, when 100 mg/kg of the dry extract of Cinnamomum cassia according to the present disclosure was administered, it was confirmed that the gastric damage area reduction and anti-inflammatory index reduction results were outstanding when compared to administering 200 and 400 mg/kg, showing outstanding effects at a specific dose, and there was no dose-dependence.

### Example 3. Clinical Trial of Acute or Chronic Gastritis Patients of Cinnamomum cassia Extract According to the Present Disclosure

The Cinnamomum cassia extract of the Preparation Example 1 according to the present disclosure, placebo, and active control drug were administered to acute and chronic gastritis patients to compare and evaluate the effectiveness and safety of Cinnamomum cassia extract according to the present disclosure in a searching manner and to determine an appropriate dose.

Specifically, acute or chronic gastritis patients with one or more erosions identified in a gastroscopy performed within 7 days from the start date of administration of investigational medical product were divided into the following test groups and administered for 2 weeks.

### Test Groups

Test group I (group administered with 225 mg of Cinnamomum cassia extract according to the present disclosure)
Test group II (group administered with 450 mg of Cinnamomum cassia extract according to the present disclosure)
Control group I (group administered with placebo)
Control group II (group administered with 180 mg of ethanol soft ext. (20->1) of 95% artemisia)
Control group III (group administered with 300 mg rebamipide)

### Effectiveness Evaluation Criteria and Evaluation Method

### 1) Primary Effectiveness Endpoint

\textcircled{1} Effective rate on gastroscopy: The proportion of test subjects whose erosion score on gastroscopy reduced by more than 50% after 2 weeks of administration (visit 3) compared to baseline (visit 1) was calculated according to the following Equation 1, and evaluated according to the criteria in Table 3.

$$Effective rate on gastroscopy ( \% ) = [ \frac{No . of effective cases}{No . of subject cases} ] \times 100 \qquad [ Equation 1 ]$$

TABLE 3

| | | Evaluation criteria of | |
|---|---|---|---|
| Evaluation criteria | | effective case of gastroscopy | |
| of endoscopy grade | | | Change in grade (before |
| | Number of | Gastroscopy | administration → after |
| Grade | erosions | decision | administration) |
| 1 | None | Effective case | 4→1, 3→1, 2→1, 4→2 |
| 2 | 1 to 2 | Ineffective case | Others |
| 3 | 3 to 5 | | |
| 4 | 6 or more | | |

### 2) Secondary Effectiveness Endpoint

\textcircled{1} Complete cure rate on gastroscopy: The proportion of test subjects who were completely cured to normal on gastroscopy (0 erosions: erosion score 1 point) after 2 weeks of administration (visit 3) compared to baseline (visit 1) was calculated and evaluated according to the following Equation 2.

$$Complete cure rate on gastroscopy ( \% ) = [ \frac{No . of effective cases}{No . of subject cases} ] \times 100 \qquad [ Equation 2 ]$$

As a result of confirming the effective rate on gastroscopy which is the proportion of the test subjects whose erosion score on gastroscopy was reduced by more than 50%, which is the primary effectiveness endpoint, the effective rate of the group with a daily dosage of 255 mg of the Cinnamomum cassia extract according to the present disclosure was the highest, and it was confirmed that effective rate was significantly higher than that of all control groups including the placebo (Table 4).

Further, the group administered with 225 mg of the Cinnamomum cassia extract according to the present disclosure showed the highest complete cure rate and showed significantly higher complete cure rate than that of all control groups including the placebo (Table 5).

225 mg of the Cinnamomum cassia extract according to the present disclosure showed the effective rate and the complete cure rate on gastroscopy that were significantly higher than those of all control groups including the placebo, and thus it was confirmed that an erosion improvement effect was excellent. However, 450 mg of the Cinnamomum cassia extract according to the present disclosure did not show a significant difference in effect from all control groups including the placebo, and thus it was confirmed that there was no dose-dependence.

TABLE 4
Primary effectiveness evaluation (effective rate on gastroscopy)

| | | | | ρ-value | |
|---|---|---|---|---|---|
| | | | | Comparison with 225 | Comparison with 450 |
| | | | | mg of Cinnamomum | mg of Cinnamomum |
| | | | Effective | cassia extract | cassia extract |
| | | | example | according to the | according to the |
| Administration group | | N | people(%) | present disclosure | present disclosure |
| Test | 225 mg of | 52 | 37(73.08%) | — | — |
| group | Cinnamomum | | | | |
| | cassia extract | | | | |
| | according to the | | | | |
| | present disclosure | | | | |
| | 450 mg of | 44 | 18(40.91%) | — | — |
| | Cinnamomum | | | | |
| | cassia extract | | | | |
| | according to the | | | | |
| | present disclosure | | | | |
| Control | Placebo | 47 | 21(44.68%) | 0.0040^{a} | 0.7164^{d} |
| group | 180 mg of ethanol | 48 | 25(52.05%) | 0.0298^{b} | 0.2832^{e} |
| | soft extract (20 → 1) | | | | |
| | of 95% artemisia | | | | |
| | 300 mg rebamipide | 42 | 20(47.62%) | 0.0116^{c} | 0.5311^{f} |

^{a}Chi-square test result for comparison between the 225 mg Cinnamomum cassia extract group and the placebo group according to the present disclosure
^{b}Chi-square test result for comparison between the 225 mg Cinnamomum cassia extract group and the 180 mg of ethanol soft ext. (20 → 1) of 95% artemisia group according to the present disclosure
^{c}Chi-square test result for comparison between the 225 mg Cinnamomum cassia extract group and the 300 mg rebamipide group according to the present disclosure
^{d}Chi-square test result for comparison between the 450 mg Cinnamomum cassia extract group and the placebo group according to the present disclosure
^{e}Chi-square test result for comparison between the 450 mg Cinnamomum cassia extract group and the 180 mg of ethanol soft ext. (20 → 1) of 95% artemisia group according to the present disclosure
^{f}Chi-square test result for comparison between the 450 mg Cinnamomum cassia extract group and the 300 mg rebamipide group according to the present disclosure

TABLE 5
Secondary effectiveness evaluation (complete cure rate on gastroscopy)

| | | | | ρ-value | |
|---|---|---|---|---|---|
| | | | | Comparison with 225 | Comparison with 450 |
| | | | | mg of Cinnamomum | mg of Cinnamomum |
| | | | Effective | cassia extract | cassia extract |
| | | | example | according to the | according to the |
| Administration group | | N | people(%) | present disclosure | present disclosure |
| Test | 225 mg of | 52 | 36(69.23%) | | — |
| group | Cinnamomum cassia | | | | |
| | extract according to | | | | |
| | the present disclosure | | | | |
| | 450 mg of | 44 | 18(40.91%) | | — |
| | Cinnamomum cassia | | | | |
| | extract according to | | | | |
| | the present disclosure | | | | |
| Control | Placebo | 47 | 20(42.55%) | 0.0075^{a} | 0.8737^{d} |
| group | 180 mg of ethanol soft | 48 | 21(43.75%) | 0.0101^{b} | 0.7830^{e} |
| | extract (20 → 1) of | | | | |
| | 95% artemisia | | | | |
| | 300 mg rebamipide | 42 | 17(40.48%) | 0.0052^{c} | 0.9674^{f} |

^{a}Chi-square test result for comparison between the 225 mg Cinnamomum cassia extract group and the placebo group according to the present disclosure
^{b}Chi-square test result for comparison between the 225 mg Cinnamomum cassia extract group and the 180 mg of ethanol soft ext. (20 → 1) of 95% artemisia group according to the present disclosure
^{c}Chi-square test result for comparison between the 225 mg Cinnamomum cassia extract group and the 300 mg rebamipide group according to the present disclosure
^{d}Chi-square test result for comparison between the 450 mg Cinnamomum cassia extract group and the placebo group according to the present disclosure
^{e}Chi-square test result for comparison between the 450 mg Cinnamomum cassia extract group and the 180 mg of ethanol soft ext. (20 → 1) of 95% artemisia group according to the present disclosure
^{f}Chi-square test result for comparison between the 450 mg Cinnamomum cassia extract group and the 300 mg rebamipide group according to the present disclosure

1. A pharmaceutical composition for preventing, improving, or treating gastritis or peptic ulcer, the pharmaceutical composition comprising
Cinnamomum cassia extract as an active ingredient, wherein a daily dosage is 210 to 360 mg.

2. The pharmaceutical composition of claim 1,
wherein the peptic ulcer is gastric ulcer.

3. The pharmaceutical composition of claim 1, wherein the Cinnamomum cassia extract is a polar solvent extract.

4. The pharmaceutical composition of claim 3, wherein the polar solvent is water.

5. The pharmaceutical composition of claim 4, wherein the water is hot water of 70° C. or higher.

6. The pharmaceutical composition of claim 3, wherein the Cinnamomum cassia extract is pretreated with a non-polar solvent before extracting with a polar solvent.

7. The pharmaceutical composition of claim 6, wherein the non-polar solvent is ethyl acetate.

8. The pharmaceutical composition of claim 1,
wherein a single dosage is 70 to 120 mg.

9. The pharmaceutical composition of claim 1,
wherein a single dosage is 70 to 80 mg.

10. The pharmaceutical composition of claim 1,
wherein the Cinnamomum cassia extract includes 5 mg/g of cinnamic acid.

11. The pharmaceutical composition of claim 1,
wherein, upon administration, an effective rate (FA set) on gastroscopy is 50% or more.

12. The pharmaceutical composition of claim 1, wherein a daily dosage is 225 mg.

13. A prevention, improvement, or treatment method of gastritis or peptic ulcer comprising administering Cinnamomum cassia extract to a subject in an amount of 210 to 360 mg per day.
